(19) United States
(12) Patent Application Publication
(10) Pub. No.: US 2005/0283022 A1
(43) Pub. Date: Dec. 22, 2005

(54) Filtrate preparation process for terephthalic acid filtrate treatment

(76) Inventor: Ronald Buford Sheppard, Kingsport, TN (US)

Correspondence Address: Dennis V. Carmen;Eastman Chemical Company, P.O. Box 511, Kingsport, TN, 37662-5075, US

(21) Appl. No.: 10/872,248

(22) Filed: Jun. 18, 2004

(51) Int. Cl. C07C 51/42

(52) U.S. Cl. 562/486

(57) ABSTRACT

A process for the production of terephthalic acid comprising: a. discharging from an oxidation reactor a crude stream comprising crude terephthalic acid solids, catalyst, impurities, and solvent, and b. without adding a fresh feed of solvent to said crude stream, separating a portion of solvent, catalyst, and at least one impurity from said crude stream to form: bi) a mother liquor composition comprising said separated solvent, catalyst, and said at least one impurity and bii) a dewatered crude terephthalic acid composition comprising a remaining portion of solvent, catalyst, impurities, and an enriched concentration of crude terephthalic acid solids relative to the solids content in the crude stream. The process has the advantage of removing a higher concentration of impurities and/or catalyst in the mother liquor stream, and/or feeding a mother liquor stream to an impurity removal and/or catalyst recovery process at a reduced flow rate, thereby reducing the size of equipment needed in such processes.

### FIELD OF THE INVENTION

The invention concerns a plant and a process for the production of terephthalic acid.

### BACKGROUND OF THE INVENTION

Terephthalic acid is produced by oxidizing para-xylene to terephthalic acid in a Co/Mn catalyst—containing solvent that includes acetic acid. After the oxidation, terephthalic acid is separated as crude solid from the by-product and catalyst-containing liquid reaction medium and suspended in a liquid that includes fresh acetic acid. The suspended solids are first separated from the contaminated acetic acid solution that is obtained as mother liquor. Afterwards, the soluble by-products are separated by extraction and distillation and eliminated and the recovered acetic acid solution and the catalyst are recycled once again to the oxidation process. The terephthalic acid product, suspended in fresh solvent, is fed to a re-oxidation and crystallization process. Afterwards, the crystalline product is dewatered and dried.

The separation of terephthalic acid as crude solid from the liquid reaction medium takes place conventionally in a centrifuge. In this process, the terephthalic acid, which is formed as a solid, is separated from the acetic acid solution in order, on the one hand, to recover the catalyst dissolved therein and, on the other hand, to eliminate by-products, which, as color-causing substances, are undesired in the final product. Usually, a washing centrifuge with a vertical shaft and with a preceding rotating filter is used and is supposed to prevent a blockage of the rotor nozzles of the centrifuge due to possible clump formation. Liquid replacement of loaded acetic acid solution by clean acetic acid takes place in the washing centrifuge. The terephthalic acid, with reduced catalyst and impurities, exits the centrifuge and arrives in the underflow tank. The solids proportion of the feed stream and typically that of the stream of purified terephthalic acid from the underflow tank are roughly the same in magnitude and amount to approximately 30 wt %. A mother liquor composition comprised of acetic acid from the crude terephthalic acid stream, wash acetic acid, catalyst and impurities exits the wash centrifuge and arrives, as mother liquor, in the overflow tank for recovery of the catalyst and removal of the impurities. Catalyst recovery and/or impurity removal can be accomplished by process steps such as but not limited to filtration, distillation, and extraction. The recovered acetic acid solution and the catalyst are recycled to the oxidation process.

It would be desirable to improve the efficiency for removal of impurities and catalyst from a mother liquor composition. We have discovered that the problem of efficiently removing catalyst and impurities in the mother liquor composition lay in both the nature of the composition itself and the flow rate of the mother liquor stream. In other words, we have found that the flow rate and the nature of the composition are significant factors affecting the efficiency of removing the catalyst and impurities from the mother liquor.

### SUMMARY OF THE INVENTION

We have discovered that a change to the flow rate of the mother liquor composition and/or a change to the mother liquor composition improve the efficiency of removing catalyst and impurities from the mother liquor composition. We have also provided a solution for simultaneously changing the mother liquor composition and changing its flow rate. There is now provided a process for the production of terephthalic acid by:
a. discharging from an oxidation reactor a crude stream comprising crude terephthalic acid solids, catalyst, impurities, and solvent, and
b. without adding a fresh feed of acetic acid to the crude stream, separating a portion of solvent, catalyst, and at least one impurity from said crude stream to form:
   bi) a mother liquor stream comprising said separated solvent, catalyst, and said at least one impurity and
   bii) a dewatered crude terephthalic acid composition comprising a remaining portion of solvent, catalyst, impurities, and an enriched concentration of crude terephthalic acid solids relative to the solids content in the crude stream
      wherein the concentration of all catalyst components in the mother liquor stream is at least 1000 ppm based on the weight of all liquids in the mother liquor stream.

In another embodiment, the mass flow rate of the mother liquor stream is smaller than those produced in conventional processes. In this embodiment:
a. a crude stream comprising crude terephthalic acid solids, catalyst, impurities, and solvent is discharged from an oxidation reactor;
b. this stream is fed into a separation device such as a centrifuge or a filter, preferably a centrifuge, or a filter at a first flow rate and separated under conditions effective to produce a:
   (bi) a mother liquor stream comprising solvent, catalyst, and impurities at a second flow rate, and
   (bii) dewatered crude terephthalic acid stream enriched in crude terephthalic acid solids relative to the solids content in the crude terephthalic acid stream fed to the separation device, preferably enriched by at least 25%, more preferably by at least 50%;
      wherein the second flow rate satisfies the following relation:
      Second Flow Rate=Q×First Flow Rate and
Q is a number within 0.2 to 0.8

In a third embodiment, terephthalic acid is made by:
a. discharging from an oxidation reactor a crude stream comprising crude terephthalic acid solids, catalyst, impurities, and solvent, and
b. feeding the crude stream to a separation device at a first flow rate and in the separation device separating a portion of solvent, catalyst, and at least one impurity from said crude stream to form:
   bi) a mother liquor stream comprising said separated solvent, catalyst, and said at least one impurity at a second flow rate; and
   bii) a dewatered crude terephthalic acid composition comprising a remaining portion of solvent, catalyst, impurities, and an enriched concentration of crude terephthalic acid solids relative to the solids content in the crude stream;
      wherein the concentration of all catalyst components in the mother liquor stream is at least 1000 ppm based on the weight of all liquids in the mother liquor stream, and the second flow rate satisfies the following relation:
      Second Flow Rate=Q×First Flow Rate and
Q is a number within 0.2 to 0.8

In yet another embodiment, there is provided a process for making terephthalic acid comprising:
a. discharging from an oxidation reactor a crude stream comprising crude terephthalic acid solids, catalyst, impurities, and solvent, and
b. without adding a fresh feed of acetic acid to the crude stream, separating under a temperature ranging from 50° C. to 200° C. and a pressure ranging from 30 to 200 psig a portion of solvent and catalyst from said crude stream within 1 minute or less to form:
   bi) a mother liquor stream comprising said separated solvent, catalyst, and said at least one impurity and
   bii) a dewatered crude terephthalic acid composition comprising a remaining portion of solvent, catalyst, impurities, and an enriched concentration of crude terephthalic acid solids relative to the solids content in the crude stream.

### BRIEF DESCRIPTION OF THE DRAWING

FIG. 1 represents a process flow diagram for a conventional method for making and purifying crude terephthalic acid.

FIG. 2 represents a process flow diagram for manufacturing and purifying terephthalic acid using a horizontally oriented decanter centrifuge.

FIG. 3 represents the downstream effect of concentrating impurities in the mother liquor when comparing a decanter centrifuge to a traditional washing disc centrifuge.

### DETAILED DESCRIPTION OF THE INVENTION

The present invention may be understood more readily by reference to the following detailed description of the invention, including the appended figures referred to herein, and the examples provided therein.

It is also be noted that, as used in the specification and the appended claims, the singular forms “a,” “an,” and “the”0 include plural referents unless the context clearly dictates otherwise. For example, reference to processing a stream is intended to include the processing of a plurality of streams.

By “comprising”0 or “containing” is meant that at least the named ingredient, or the named process parameter, or the named apparatus must be present apparatus and process, but does not exclude the presence of other ingredients, process parameters, or equipment, even if the other such other ingredients, process parameters, or equipment, have the same function as what is named. Moreover, the word comprising leaves open the possibility of inserting or using process steps or equipment before, after, or in between any of the named steps or equipment.

As used throughout the specification and claims, feeding a stream to a named vessel or from one named vessel to another named vessel does not limit the feed to a direct feed, intervening process steps and apparatus, and does not exclude the possibility that the stream composition is altered en route to the named vessel. For example, a crude terephthalic acid stream may be fed through any one or a combination of an underflow tank, one or more post-oxidation reactors, and/or one or more crystallizers, before reaching the separation vessel used in the claimed process.

Ranges include any integers and fractions thereof between the stated range, and includes the end points of the stated range. Stating that a range is at least a certain number includes numbers greater than the one stated. Stating that a range is no greater than a certain number includes numbers less than the one stated.

The process of the invention results in the production of a mother liquor stream having a smaller flow rate and a smaller mass (solids and liquids) from the separation device relative to the mother liquor flow from a washing separation device, thereby allowing for the use of smaller equipment for comparable removal/recovery of catalyst and impurities from the mother liquor stream or increased removal/recovery at equivalent mother liquor stream flow rates.

In a conventional process, a fresh feed of solvent is used as a washing medium in a washing disc centrifuge to produce a mother liquor composition that is diluted with the washing medium (e.g. acetic acid). A washing separation device uses a washing medium such as acetic acid fed to the device to separate a portion of catalyst and impurities from crude terephthalic acid solids. This results in a mother liquor stream that has a high flow rate, necessitating the use of larger size downstream purification and/or recovery equipment to handle the mass flow, and/or a mother liquor stream that has low concentration of catalyst components. In the process of the invention, a mother liquor composition is produced which is either:
a. more concentrated in catalyst and/or impurities at the same mass flow as a mother liquor stream from a washing disc centrifuge, or
b. has a much smaller mass flow at the same catalyst and/or impurity concentration compared to the mother liquor stream from a washing disc centrifuge, or
c. a combination of a) and b).

This result is achieved by reducing the amount of and preferably eliminating the use of a washing stream in the separation process, and by controlling the operational parameters of the separation device, and by the appropriate selection of the separation device used.

In one embodiment, terephthalic acid is produced by:
a. discharging from an oxidation reactor a crude stream comprising crude terephthalic acid solids, catalyst, impurities, and solvent, and
b. without adding a fresh feed of solvent to the crude stream, separating a portion of solvent, catalyst, and at least one impurity from said crude stream to form:
   bi) a mother liquor stream comprising said separated solvent, catalyst, and said at least one impurity and
   bii) a dewatered crude terephthalic acid composition comprising a remaining portion of solvent, catalyst, impurities, and an enriched concentration of crude terephthalic acid solids relative to the solids content in the crude stream
      wherein the concentration of all catalyst components in the mother liquor stream is at least 1000 ppm based on the weight of all liquids in the mother liquor stream. Alternatively, the concentration of all impurities in the mother liquor stream is at least 500. In either case, the concentration of all the catalyst component and/or impurities is at least 1300, or at least 1700 as discharged from the separation device without further processing as in, for example, flash vessels.

In another embodiment, the mass flow rate of the mother liquor stream is smaller than those produced in conventional processes. In this embodiment:
a. a crude stream comprising crude terephthalic acid solids, catalyst, impurities, and solvent is discharged from an oxidation reactor;
b. this stream is fed into a separation device such as a centrifuge or a filter, preferably a centrifuge, or a filter at a first flow rate and separated under conditions effective to produce a:
   (bi) a mother liquor stream comprising solvent, catalyst, and impurities at a second flow rate, and
   (bii) dewatered crude terephthalic acid stream enriched in crude terephthalic acid solids relative to the solids content in the crude terephthalic acid stream fed to the separation device, preferably enriched by at least 25%, more preferably by at least 50%;
      wherein the second flow rate satisfies the following relation:
      Second Flow Rate=Q×First Flow Rate and
Q is a number within 0.2 to 0.8

Since it is desirable to reduce the size of purification equipment used to process the mother liquor stream, Q is desirably 0.7 or less, or 0.6 or less.

In a third embodiment, there is provided a combination of the first two embodiments to produce a mother liquor stream that is both concentrated in catalyst and/or impurities, and has a second small flow rate relative to the first flow rate of the crude stream feeding the separation device. In this third embodiment of the invention, terephthalic acid is made by:
a. discharging from an oxidation reactor a crude stream comprising crude terephthalic acid solids, catalyst, impurities, and solvent, and
b. feeding the crude stream to a separation device at a first flow rate and in the separation device separating a portion of solvent, catalyst, and at least one impurity from said crude stream to form:
   bi) a mother liquor stream comprising said separated solvent, catalyst, and said at least one impurity at a second flow rate; and
   bii) a dewatered crude terephthalic acid composition comprising a remaining portion of solvent, catalyst, impurities, and an enriched concentration of crude terephthalic acid solids relative to the solids content in the crude stream;
      wherein the concentration of all catalyst components in the mother liquor stream is at least 1000 ppm based on the weight of all liquids in the mother liquor stream, and the second flow rate satisfies the following relation:
      Second Flow Rate=Q×First Flow Rate and
is a number within 0.2 to 0.8

In each of these embodiments, the invention realizes a more effective downstream catalyst/impurity removal process by supplying a mother liquor composition more concentrated in catalyst and impurities; the downstream equipment can be reduced in size and scope; or both.

Process conditions effective to generate enriched dewatered streams and more concentrated and/or a lower mass flow mother liquor stream within the separation device are conducting the separation at a temperature within a range of 50° C. to 200° C. Desirably, the temperature of the crude terephthalic acid stream in the separation device or the temperature applied to the crude terephthalic acid stream in the separation device is +/−30° C., or +/−15° C. of the crude terephthalic acid stream temperature discharged from the primary oxidation vessel. The pressure within the separation device is within a range of 30 psig to 200 psig in order to prevent excessive vaporation of solvent and precipitation of impurities.

The crude stream discharged from an oxidation reactor generally contains crude terephthalic acid solids, catalyst, impurities, and solvent. The crude stream is fed directly or indirectly into a means for separating solids from liquids, and then discharged from the separation means as a dewatered crude terephthalic acid stream enriched in crude terephthalic acid solids relative to the solids content in the crude terephthalic acid stream fed to the centrifuge.

As noted above, the feed of crude terephthalic acid stream effluent from the oxidation reactor to the separation device can be direct or indirect through other vessels, such as a holding tank to even out pulsations in the stream flow. Moreover, any other equipment which changes the composition of the crude terephthalic acid stream may be located between the oxidation reactor and the centrifuge.

The crude terephthalic acid stream discharged from the oxidation reactor contains crude terephthalic acid solids which may actually be in a solid precipitated form or dissolved in the solvent or as a mixture of the two. The stated solids content can be measured by precipitating out all the crude terephthalic acid in the stream being analyzed. The crude terephthalic acid stream also contains impurities. Examples of impurities include 4-carboxy benzaldehyde, p-toluic acid, benzoic acid, iso-phthalic acid, and fluorenones. The crude terephthalic acid stream also contains catalyst, optional promoters such as bromine, and the solvent.

The catalyst system may comprise a source of zirconium atoms, nickel atoms, manganese atoms, cobalt atoms, bromine atoms, and/or a source of pyridine. The source of metals may be provided in the form of metal salts, such as their nitrates, halides, borates, or their cationic salts of aliphatic or aromatic acids having 2-22 carbon atoms. The bromine component may be added as elemental bromine, in combined form or as an anion. Suitable sources of bromine include hydrobromic acid, sodium bromide, ammonium bromide, potassium bromide, tetrabromoethane, benzyl bromide, 4-bromopyridine, alpha-bromo-p-toluic acid, and bromoacetic acid.

In general, suitable amounts of catalyst components (not their compound weight) in the oxidation reactor liquid phase range from 1000 ppm to 9000 ppm of total combined metal and bromine atoms, although more or less can be used if desired, especially as the oxidation reaction temperature is changed. The weight amount of each of the catalyst components is based on the atomic weight of the atom, whether or not the atom is in elemental form or in ionic form.

The liquid phase oxidation reaction in the primary oxidation reactor is generally carried out in the presence of a solvent. Suitable solvents include water and the aliphatic solvents. The preferred aliphatic solvents are aliphatic carboxylic acids which include, but are not limited to, aqueous solutions of C₂ to C₆ monocarboxylic acids, e.g., acetic acid, propionic acid, n-butyric acid, isobutyric acid, n-valeric acid, trimethylacetic acid, caprioic acid, and mixtures thereof. Preferably, the solvent is volatile under the oxidation reaction conditions to allow it to be taken as a vapor from the oxidation reactor. It is also preferred that the solvent selected is also one in which the catalyst composition is soluble under the reaction conditions.

The most common solvent used for the oxidation of p-xylene is an aqueous acetic acid solution, typically having a concentration of 80 to 99 wt. % acetic acid. In especially preferred embodiments, the solvent comprises a mixture of water and acetic acid which has a water content of about 2.5% to about 15% by weight. A portion of the solvent feed to the primary oxidation reactor may be obtained from a recycle stream obtained from the solvent contained in the mother liquor stream after the crude terephthalic acid stream is separated.

The crude terephthalic acid stream discharged from the oxidation reactor is fed to the separation device at a first flow rate directly, or indirectly through any type or number of vessels, such as underflow tanks, post-oxidation reactors, and/or crystallizers. Without adding a fresh feed of solvent such as acetic acid to the crude terephthalic acid stream in the separation device, a portion of solvent (e.g. acetic acid), catalyst, and impurities is separated from the crude stream to form a mother liquor composition comprising said separated solvent, catalyst, and impurities and a dewatered crude terephthalic acid composition comprising a remaining portion of solvent, catalyst, impurities, and an enriched concentration of crude terephthalic acid solids relative to the solids content in the crude stream. The particular amount of solvent, catalyst, and impurities separated from the stream is not limited, although it is desirable to separate as much of these ingredients into the mother liquor stream as possible so as to maximize their recovery in one step and efficiently purify the stream in one step.

It is desirable to separate the crude terephthalic acid stream to produce a mother liquor stream highly concentrated in catalyst and/or impurities. The catalyst concentration in the mother liquor stream is based on the weight of all catalyst components relative to the weight of all liquids in the mother liquor stream. Examples of catalyst components are the same examples of catalyst components identified above as used in the primary oxidation reactor, based on their atom weight. The concentration of all catalyst components in the mother liquor stream is preferably at a concentrated level of at least 1000 ppm, or at least 1500 ppm, or at least 2000 ppm, based on the weight of all liquids in the mother liquor stream as discharged from the separation device.

Likewise, the concentration of all impurities in the mother liquor stream is at least 1500 ppm. The concentration of impurities is based on the compound weight of the impurity in the mother liquor stream discharged from the separation device.

The dewatered crude terephthalic acid composition comprising a remaining portion of solvent, catalyst, and impurities is enriched in the concentration of crude terephthalic acid solids. Some solvent remains in the dewatered terephthalic acid stream due to separation limitations of the equipment. As a result of the separation process, however, the dewatered crude terephthalic acid stream is enriched in crude terephthalic acid solids relative to the solids concentration in the crude terephthalic acid stream fed to the separation device. Preferably, the degree of enrichment is at least 25%, more preferably at least 50%, and even 100% or more, or 150% or more, or 200% or more. As above, the degree of enrichment is calculated as:

$$\frac{\begin{matrix} \% solids in dewatered stream - \\ \% solids in crude stream\end{matrix}}{\% solids in crude stream} \times 100$$

The separation device is in fluid communication with the oxidation reactor. The fluid communication may be direct or indirect through a one or more vessels or processes. The separation device has at least an inlet to receive the crude terephthalic acid stream, a separator for separating a portion of the solvent and catalyst from the crude stream to form the mother liquor composition and the dewatered terephthalic acid stream enriched in solid relative to the solid concentration in the crude stream, and outlets for discharging the dewatered terephthalic acid stream and the mother liquor composition.

Examples of suitable separation devices include centrifuges and filters. The preferred centrifuge is a decanter centrifuge. Both vertical and horizontal centrifuges are acceptable in this application. As noted above, conditions suitable for providing the enriched dewatered terephthalic acid stream and the dewatered mother liquor stream include operating the separation device between about 50° C. to about 200° C., preferably 140° C. to about 170° C. and at pressures between about 30 psig to about 200 psig. An example of a filter is a Pannevis filter. The residence time can be any residence time suitable to remove a portion of the solvent and produce a slurry product. Desirably, the residence time of the crude terephthalic acid stream in the separation device is 1 minute or less. The residence time is the average time that a hypothetical marker in the crude terephthalic acid stream at the inlet of the separation device travels through the separation device and is discharged either through the mother liquor stream outlet or the dewatered terephthalic acid stream outlet. The centrifuge or filter may be operated in the continuous or batch mode, preferably in the continuous mode.

Accordingly, in another embodiment of the invention, a process for making terephthalic acid comprises:
a. discharging from an oxidation reactor a crude stream comprising crude terephthalic acid solids, catalyst, impurities, and solvent, and
b. without adding a fresh feed of solvent to the crude stream, separating under a temperature ranging from 50° C. to 200° C. and a pressure ranging from 30 to 200 psig a portion of solvent and catalyst from said crude stream within 1 minute or less to form:
   bi) a mother liquor stream comprising said separated solvent, catalyst, and said at least one impurity and
   bii) a dewatered crude terephthalic acid composition comprising a remaining portion of solvent, catalyst, impurities, and an enriched concentration of crude terephthalic acid solids relative to the solids content in the crude stream.

In one embodiment, at least 50% of the catalyst is separated and removed, and more preferably at least 85% of the catalyst is removed from the crude terephthalic acid stream and into the mother liquor stream. Also, at least 50% of the impurities can be separated and removed, and more preferably at least 85% of the impurities are removed from the crude stream and into the mother liquor stream. The remainder of the catalyst and impurities are in the dewatered crude terephthalic acid stream.

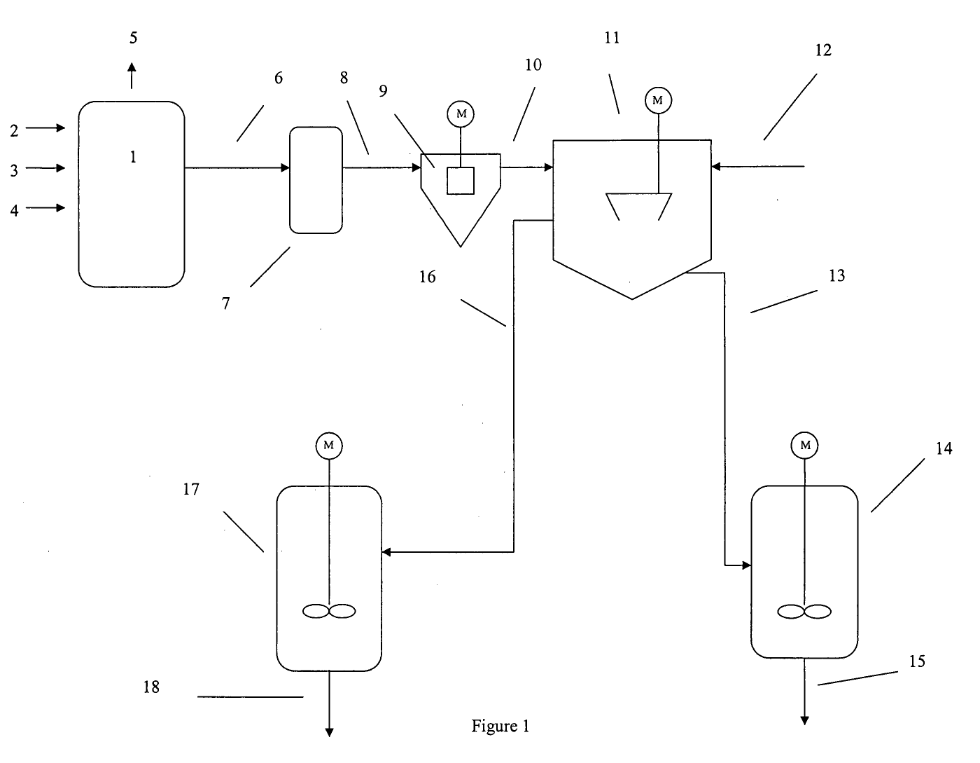

A typical washing disc centrifuge, which has been employed in this process in the past, is displayed in FIG. 1. In FIG. 1, para-xylene is fed via line (2), oxygen via line (3), and acetic acid and catalyst via line (4) into a reactor (1). After the transformation to crude terephthalic acid, water vapor and acetic acid vapor are drawn from the reactor (1) through line (5) and crude terephthalic acid, as crude solid, which, together with catalyst material and impurities dissolved in acetic acid, along with residual water is passed via line (6) first into a collecting tank (7), which evens out fluctuations. Afterwards, the crude terephthalic acid is fed via line (8) into a rotating filter (9) and then via line (10) into a washing centrifuge (11). The rotating filter (9) prevents a blockage of the rotor nozzles of the washing centrifuge (11) in the event of possible clumping of the crude terephthalic acid. The proportion of crude terephthalic acid solids that is drawn off via line (6) amounts, in this illustrative solution, to approximately 30 wt %. A liquid exchange between loaded acetic acid contained in the crude terephthalic acid stream through line 10, and fresh acetic acid, which is fed via line (12) into the washing centrifuge (11), takes place in the washing centrifuge (11). The crude terephthalic acid, continuing to have a solids concentration of about 30%, is passed via line (13) first into an underflow tank (14) and from there, via line (15) to a post-oxidation reactor, which is not shown. The treatment of the material originating in line (13) is not limited to a post-oxidation step; other unit operations can be performed.

During the separation of the media in the washing centrifuge (11), the mother liquor acetic acid solution, loaded with catalyst material and impurities, is passed via line (16) into an overflow tank (17) and from there, via line (18), to a filtrate treatment unit, which is not shown, for recovery of the catalyst material, removal of impurities, and recovery of the acetic acid. During the washing exchange inside the centrifuge, the liquor fed to the centrifuge as part of the slurry (10) is naturally diluted with the fresh acetic acid (12). This results in ratios of fresh acetic acid/liquor feed to the centrifuge of 0.1 to 1.5, preferably of 0.3 to 1.1. The mass of the mother liquor stream (liquid and solids) can be equal to or greater than the fresh acetic acid/liquor feed ratio due to the supply of fresh feed to the washing centrifuge. An illustration will follow after description of the washing and decanter centrifuges below.

In contrast to washing centrifuges, a decanter centrifuge operates with a solid bowl, which rotates around a horizontal or vertical axis and contains a spiral-shaped screw conveyer in order to separate the solid-liquid mixture feed into its solid and liquid components. Alternatively, a decanter centrifuge can also operate with a screen solid bowl, in which case the solids, prior to their exit from the conveyer, are pressed through an additionally perforated screen section of the solid bowl.

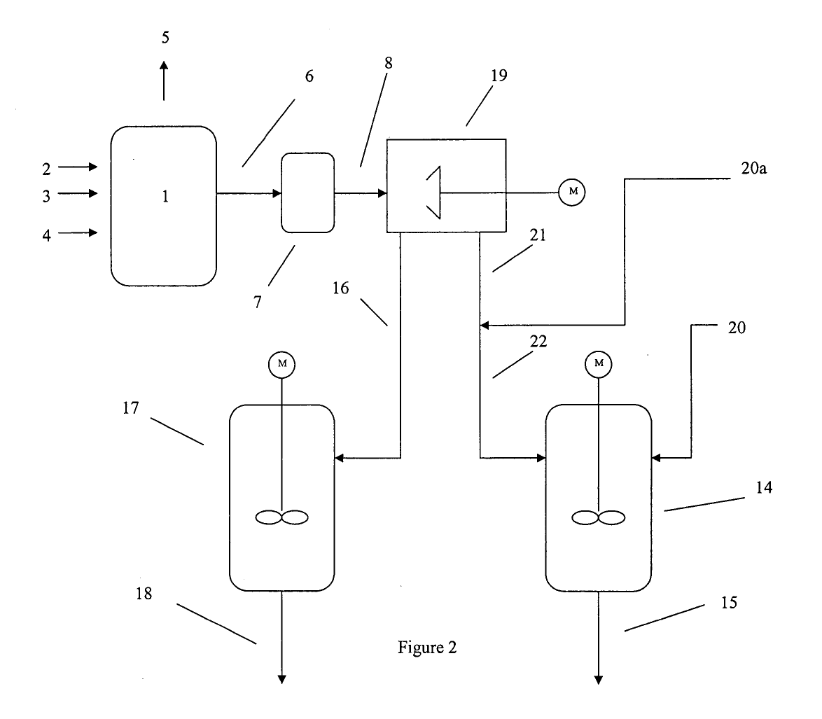

FIG. 2 illustrates an embodiment of the invention. P-xylene is fed via line (2), oxygen via line (3), and acetic acid and catalyst via line (4) into a reactor (1). After the transformation to crude terephthalic acid, water vapor and acetic acid vapor are drawn from the reactor (1) through line (5) and crude terephthalic acid, as crude solid, which together with catalyst material and impurities dissolved in acetic acid, along with residual water, is passed via line (6) first into an optional collecting tank (7) to regulate fluctuations in flow. Afterwards, the crude terephthalic acid stream is discharged from the optional collecting tank (7) through line (8) and fed to a decanter centrifuge (19), or discharged from the reactor 1 through line 6 and fed directly into a decanter centrifuge (19). In this embodiment, the crude terephthalic acid stream is fed into a centrifuge without passing through a rotating filter.

In the decanter centrifuge (19), the crude terephthalic acid stream is dewatered to a residual moisture content of about 20 wt %. The dewatered crude terephthalic acid stream, now with a solids content of at least about 50% and up to about 85 wt %, is passed from the decanter centrifuge (19) via line (21 and 22) into a receiver, pipe or tank (14), into which fresh acetic acid is fed via line (20). Alternatively, the fresh acetic acid can be fed in via line (20a) directly at the outlet of the decanter centrifuge (19) into line (22) instead of feeding acetic acid through line 20. If desired, fresh acetic acid may be fed to the dewatered crude terephthalic acid stream through lines 20 and 20a. Feeding fresh acetic acid through line 20a between the horizontal decanter centrifuge and the receiver (14) has the advantage that it avoids possible solid blockages. The dewatered crude terephthalic acid is thereby mixed with the clean acetic acid to a solids content ranging from 15 to 50 wt. % to form a purified terephthalic acid composition. For example, the solids content can be 30 wt %. The crude terephthalic acid is passed from the receiver (14) via line (15) to a post-oxidation reactor, which is not shown. The treatment of the material originating in line (21) is not limited to a post-oxidation step; other unit operations can be performed.

During the separation of the media in the decanter centrifuge (19), part or all of the acetic acid mother liquor, loaded with catalyst material and impurities, is passed via line (16) into a receiver, pipe or tank (17) and from there, via line (18), to a flash cooling and impurity removal process, which is not shown. An example of an impurity removal process may be found in U.S. Pat. No. 4,939,297, incorporated herein by reference in its entirety.

In contrast with FIG. 1, the acetic acid in the crude terephthalic acid stream is not exchanged with fresh acetic acid, and the addition of fresh acetic acid does not take place in the centrifuge bowl. Rather, the mother liquor acetic acid is first separated in the decanter centrifuge bowl and discharged through line 16 into an overflow tank. The dewatered crude terephthalic acid stream is diluted with feed of fresh replacement acetic acid outside the centrifuge bowl at the discharge housing or downstream from the centrifuge. Thus, in a process of the invention, mother liquor acetic acid comprising acetic acid and catalyst is separated from a crude terephthalic acid stream to form a dewatered crude terephthalic acid stream having an enriched concentration of crude terephthalic acid solids relative to the solids concentration of the crude terephthalic acid stream, followed by diluting the dewatered crude terephthalic acid stream with a fresh feed of acetic acid to reduce the solids concentration.

The mother liquor is further treated to remove impurities generated in the oxidation step and recover the catalyst. A further advantage of a decanter centrifuge for the process herein is that, unlike the washing centrifuge, the mother liquor produced and fed to the extraction process for impurity removal is more concentrated in impurities. This is now further illustrated with an example.

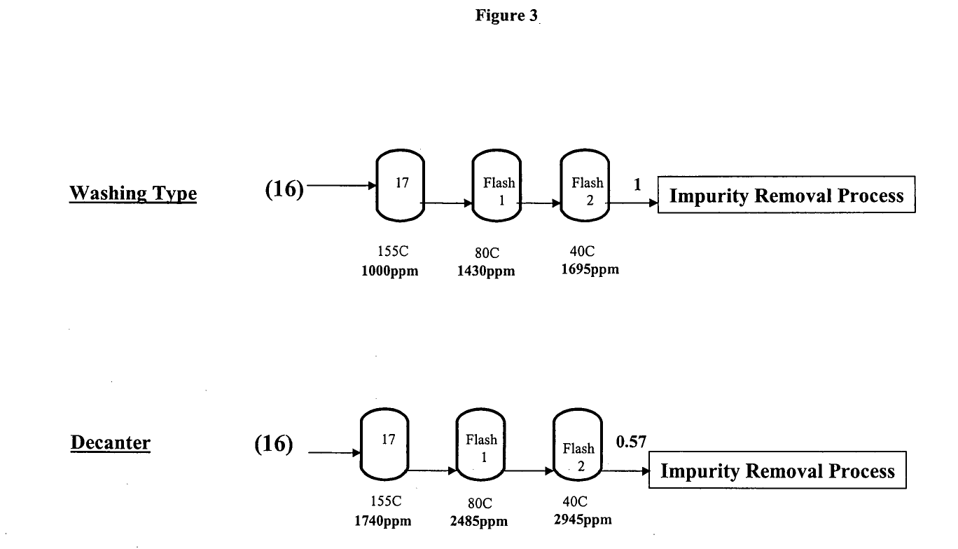

FIG. 3 provides an illustration of the difference on downstream equipment size requirements between the traditional washing centrifuge and the decanter. The mother liquor from the respective centrifuges is represented by stream (16) being fed to holdup tank (17), which correspond to the respective streams in FIGS. 1 and 2. In preparation for impurity removal, the streams are stepwise cooled using two flash stages (Flash 1 and Flash 2) as indicated. During the flash stages, solvent is removed as a vapor from the mother liquor which results in further concentration of impurities in the mother liquor feeding the impurity removal process. The case illustrated in FIG. 3 shows the relative effect of dilution that results from a washing centrifuge in comparison to a decanter centrifuge. The ppm level shown for each vessel shows an example ppm level of an impurity such as isophthalic acid or a catalyst such as cobalt that is removed and/or recovered in a downstream filtrate treatment process. The concentration of impurity or catalyst in stream 16 fed from the washing centrifuge to the flash stage in the process of FIG. 1 is diluted (1000 ppm) when compared to concentration of impurity or catalyst in stream (16) from the decanter centrifuge (1740 ppm) because in the former, a fresh feed of acetic acid was used as a washing medium. Following the flow in the washing centrifuge case, the stream having a concentration of 1000 ppm in the holdup vessel operated at 140-190° C. is fed to a first flash vessel operated at less than 110° C. to concentrate the impurity to 1430 ppm, followed by feeding the stream to a second flash vessel operated at less than 80° C. to concentrate the impurity to 1695 ppm. By contrast, in the decanter centrifuge case, the stream having a concentration of 1740 ppm in the holdup vessel operated at 140-190° C. is fed to a first flash vessel operated at less than 110° C. to concentrate the impurity to 2485 ppm, followed by feeding the stream to a second flash vessel operated at less than 80° C. to concentrate the impurity to 2945 ppm. In this example the temperatures of the holdup vessel, first flash vessel, and second flash vessel are the same for both the washing centrifuge and decanter centrifuge cases.

It is apparent that the concentration of catalysts and/or impurities in the feed from the decanter centrifuge is significantly higher than the washing centrifuge. In fact, to remove the same amount of impurities from the process, for the decanter centrifuge one would only need to feed the impurity removal process with 57% of the feed required using the washing centrifuge as the feed source. This can be illustrated by simple material balance taking the wash centrifuge as the base case:
Wash centrifuge−Feed Rate=1, Impurity concentration=1695
Decanter centrifuge−Feed Rate=X, Impurity concentration=2945

Solving for feed rate (X) to the decanter centrifuge, keeping total impurity level fed to the impurity removal process constant, 57% of the feed for the decanter centrifuge is required (0.57): X=(1*1695)/2945=0.57

This has a direct impact not only on the size of the flash stages to produce the concentrated mother liquor, but also the downstream equipment located inside the impurity removal process, thereby improving the overall cost of the plant.

An alternative approach is to also feed the same amount of mother liquor to the holdup tank and impurity removal process to thereby remove more impurities from the process, which results in a more purified terephthalic acid product for the same size of impurity removal process and a larger quantity of impurity and catalyst recovered in this step. As an example, assuming that the efficiency of impurities removed in the impurity removal process is 100% for the same feed rate of mother liquor for the two type of centrifuges, the following analysis can be made. For the same feed rate to the impurity removal process, the decanter case will remove 74% more impurities compared to the wash centrifuge case. This is illustrated below:
Wash centrifuge−Feed Rate=1, Impurity concentration=1695
Decanter centrifuge−Feed Rate=1, Impurity concentration=2945
% Increase in Impurities Removed=(2945−1695)/1695*100%=74%. The percentage increase in impurity removal, catalyst removal, or both can be at least 1%, more preferably at least 25%, and most preferably at least 50%.

A further advantage of the process of the invention results from the fact that the rotating filters that are arranged before the washing centrifuge in accordance with prior art can now be dispensed with if desired. Since the decanter centrifuge (19) does not have rotor nozzles, preceding rotating filters are no longer needed.

1. A process for the production of terephthalic acid comprising:
a. discharging from an oxidation reactor a crude stream comprising crude terephthalic acid solids, catalyst, impurities, and solvent, and
b. without adding a fresh feed of acetic acid to the crude stream, separating a portion of solvent, catalyst, and at least one impurity from said crude stream to form:
bi) a mother liquor stream comprising said separated solvent, catalyst, and said at least one impurity and
bii) a dewatered crude terephthalic acid composition comprising a remaining portion of solvent, catalyst, impurities, and an enriched concentration of crude terephthalic acid solids relative to the solids content in the crude stream
wherein the concentration of all catalyst components in the mother liquor stream is at least 1000 ppm based on the weight of all liquids in the mother liquor stream.

2. The process of claim 1, wherein the concentration of catalyst (in ppm) in the mother liquor stream relative to the liquids in the mother liquor stream is higher than the concentration of catalyst (in ppm) in the crude stream relative to the liquids and solids in the crude stream.

3. The process of claim 1, wherein the concentration of at least one impurity (in ppm) in the mother liquor stream relative to the liquids in the mother liquor stream is higher than the concentration of the same impurity (in ppm) in the crude stream relative to the liquids and solids in the crude stream.

4. The process of claim 1, wherein the concentration of catalyst and at least one impurity (each in ppm) in the mother liquor stream relative to the liquids in the mother liquor stream is higher than the concentration of the same catalyst and impurity (each in ppm) in the crude stream relative to the liquids and solids in the crude stream.

5. The process according to any one of claims 2-4, wherein the degree of enrichment is at least 25%.

6. The process according to any one of claims 2-4, wherein the degree of enrichment is at least 50%.

7. The process according to claim 1, wherein the degree of enrichment of solids in the dewatered stream is at least 25%.

8. The process according to claim 1, wherein the degree of enrichment of solids in the dewatered stream is at least 50%.

9. The process according to claim 1, wherein the concentration of solid in the dewatered stream ranges from 50% to 85%.

10. A process for making terephthalic acid comprising:
a. discharging a crude stream comprising crude terephthalic acid solids, catalyst, impurities, and solvent from an oxidation reactor;
b. feeding said crude stream into a separation device at a first flow rate and separating the crude stream under conditions effective to produce a:
(bi) a mother liquor stream comprising solvent, catalyst, and impurities at a second flow rate, and
(bii) dewatered crude terephthalic acid stream enriched in crude terephthalic acid solids relative to the solids content in the crude terephthalic acid stream fed to the separation device, preferably enriched by at least 25%, more preferably by at least 50%;
wherein the second flow rate satisfies the following relation:
Second Flow Rate=Q×First Flow Rate and
Q is a number within 0.2 to 0.8

11. The process of claim 10, wherein the separation device is a centrifuge.

12. The process of claim 11, wherein the centrifuge is a decanter centrifuge.

13. The process of claim 12, wherein the centrifuge is a horizontal decanter centrifuge.

14. The process of claim 10, wherein the separation device is a filter.

15. The process of claim 14, wherein the filter is a Pannevis filter.

16. The process according to claim 10, wherein the degree of enrichment of solids in the dewatered stream is at least 25%.

17. The process according to claim 10, wherein the degree of enrichment of solids in the dewatered stream is at least 50%.

18. The process of claim 10, wherein the concentration of catalyst (in ppm) in the mother liquor stream relative to the liquids in the mother liquor stream is higher than the concentration of catalyst (in ppm) in the crude stream relative to the liquids and solids in the crude stream.

19. The process of claim 10, wherein the concentration of at least one impurity (in ppm) in the mother liquor stream relative to the liquids in the mother liquor stream is higher than the concentration of the same impurity (in ppm) in the crude stream relative to the liquids and solids in the crude stream.

20. The process of claim 10, wherein the concentration of catalyst and at least one impurity (each in ppm) in the mother liquor stream relative to the liquids in the mother liquor stream is higher than the concentration of the same catalyst and impurity (each in ppm) in the crude stream relative to the liquids and solids in the crude stream.

21. A process for making terephthalic acid comprising:
a. discharging from an oxidation reactor a crude stream comprising crude terephthalic acid solids, catalyst, impurities, and solvent, and
b. feeding the crude stream to a separation device at a first flow rate and in the separation device separating a portion of solvent, catalyst, and at least one impurity from said crude stream to form:
bi) a mother liquor stream comprising said separated solvent, catalyst, and said at least one impurity at a second flow rate; and
bii) a dewatered crude terephthalic acid composition comprising a remaining portion of solvent, catalyst, impurities, and an enriched concentration of crude terephthalic acid solids relative to the solids content in the crude stream;
wherein the concentration of all catalyst components in the mother liquor stream is at least 1000 ppm based on the weight of all liquids in the mother liquor stream, and the second flow rate satisfies the following relation:
Second Flow Rate=Q×First Flow Rate and
Q is a number within 0.2 to 0.8

22. The process of claim 21, wherein the separation device is a centrifuge.

23. The process of claim 22, wherein the centrifuge is a decanter centrifuge.

24. The process of claim 23, wherein the centrifuge is a horizontal decanter centrifuge.

25. The process of claim 21, wherein the separation device is a filter.

26. The process of claim 25, wherein the filter is a Pannevis filter.

27. The process according to claim 21, wherein the degree of enrichment of solids in the dewatered stream is at least 25%.

28. The process according to claim 21, wherein the degree of enrichment of solids in the dewatered stream is at least 50%.

29. A process for making terephthalic acid comprising:
a. discharging from an oxidation reactor a crude stream comprising crude terephthalic acid solids, catalyst, impurities, and solvent, and
b. without adding a fresh feed of acetic acid to the crude stream, separating under a temperature ranging from 50° C. to 200° C. and a pressure ranging from 30 to 200 psig a portion of solvent and catalyst from said crude stream within 1 minute or less to form:
bi) a mother liquor stream comprising said separated solvent, catalyst, and said at least one impurity and
bii) a dewatered crude terephthalic acid composition comprising a remaining portion of solvent, catalyst, impurities, and an enriched concentration of crude terephthalic acid solids relative to the solids content in the crude stream

30. The process of claim 29, wherein the mother liquor composition comprises acetic acid and catalysts effective for liquid phase oxidation of p-xylene to terephthalic acid in the presence of oxygen.

31. The process of claim 29, wherein the flow rate of the mother liquor composition is at least 25% less.
